(19) United States
(12) Patent Application Publication
(10) Pub. No.: US 2025/0381368 A1
(43) Pub. Date: Dec. 18, 2025

(54) SHEATH HOLDER AND RELATED SYSTEMS AND METHODS

(71) Applicant: Merit Medical Systems, Inc., South Jordan, UT (US)

(72) Inventors: Brendan Knight, South Jordan, UT (US); Aaron Hopkinson, Herriman, UT (US); Janet Hudnall, South Jordan, UT (US); Tab Robinson, South Jordan, UT (US); Manit Sabharwal, South Jordan, UT (US); Tom Meese, South Jordan, UT (US); Garrett Myers, South Jordan, UT (US); Adam Simmons, South Jordan, UT (US); Kevin Sterba, South Jordan, UT (US)

(21) Appl. No.: 19/234,932

(22) Filed: Jun. 11, 2025

Related U.S. Application Data
(60) Provisional application No. 63/660,410, filed on Jun. 14, 2024.

(51) Int. Cl. A61M 25/02

(52) U.S. Cl. CPC A61M 25/02; A61M 2025/024; A61M 2025/028; A61M 2205/0216; A61M 2209/088

(57) ABSTRACT

A sheath holder is provided, including a clip including a first receptacle configured to secure a sheath or hub of an introducer system to the clip, and a first anchoring portion. The sheath holder further includes a base configured to secure the first anchoring portion of the clip to an exterior surface of a body of a patient.

### RELATED APPLICATIONS

This application claims priority to U.S. Provisional Patent Application No. 63/660,410, filed on Jun. 14, 2024 and titled, “Sheath Holder and Related Systems and Methods,” which is hereby incorporated by reference in its entirety.

### TECHNICAL FIELD

The present disclosure relates generally to the field of introducer sheaths. More particularly, some embodiments relate to methods for securing introducer sheaths.

### BRIEF DESCRIPTION OF THE DRAWINGS

The written disclosure herein describes illustrative embodiments that are non-limiting and non-exhaustive. Reference is made to certain of such illustrative embodiments that are depicted in the figures, in which:

FIG. 1 illustrates a perspective view of an introducer system, according to embodiments described herein.

FIG. 2 illustrates a perspective view of an attachment assembly, according to embodiments described herein.

FIG. 3 illustrates a perspective view of a clip of an attachment assembly, according to embodiments described herein.

FIG. 4 illustrates a perspective view of an attachment portion of an introducer system, according to embodiments described herein.

FIG. 5 illustrates a top-down view of an attachment assembly and an attachment portion of an introducer system, according to embodiments disclosed herein.

FIG. 6 illustrates a lateral view of an insertion point of an introducer system, according to embodiments disclosed herein.

FIG. 7 illustrates an attachment assembly for use with an introducer system, according to embodiments described herein.

FIG. 8 illustrates a perspective view of a clip of an attachment assembly, according to embodiments described herein.

FIG. 9 illustrates a perspective view of a clip for use with an introducer system, according to embodiments described herein.

FIG. 10 illustrates a lateral view of an insertion point of an introducer system including the clip of FIG. 9, according to embodiments disclosed herein.

### DETAILED DESCRIPTION

Introducer sheaths are inserted into vasculature such as the radial or femoral artery to facilitate the introduction of medical instruments (catheters, guide wires, etc.), during various procedures. These sheaths serve as a conduit to a patient's vasculature. By creating a controlled entry point this helps to minimize trauma to the vessel walls and reduce the risk of complications.

One common problem with conventional introducer sheaths is that they tend to slip or back out of the vasculature during medical procedures. Slippage can occur due to various factors, such as patient movement, blood flow dynamics, or friction between the sheath and vessel walls. When a sheath backs out, the procedure can be disrupted, potentially causing delays and complications. This increases the risk of vessel damage and patient discomfort, as well as necessitates additional maneuvers to reinsert and stabilize the sheath, prolonging the procedure time. Furthermore, the need for repeated adjustments can lead to increased exposure to radiation for both the patient and the medical team (for instance, in procedures guided by fluoroscopy).

Additionally, when adhesion is used to fix components, and is positioned too close to the insertion site, fluids that leak from the insertion site can compromise the integrity of the adhesive bond. Reduction in adhesion strength can result in the fixation component becoming loose or dislodged, thereby compromising the stability and security of the medical device, or sheath it is meant to hold in place.

Aspects and implementations of the present disclosure address these and other challenges by providing systems and methods for stabilizing a sheath, such as through adhesive fixation, in a way that reduces the risk of sheath or introducer slippage, as well as using methods that protect that integrity of the adhesive bond.

The components of the embodiments as generally described and illustrated in the figures herein can be arranged and designed in a wide variety of different configurations. Thus, the following more detailed description of various embodiments, as represented in the figures, is not intended to limit the scope of the present disclosure but is merely representative of various embodiments. While various aspects of the embodiments are presented in drawings, the drawings are not necessarily drawn to scale unless specifically indicated.

The phrase “coupled to” is broad enough to refer to any suitable coupling or other form of interaction between two or more entities, including mechanical interaction. Thus, two components may be coupled to each other even though they are not in direct contact with each other. The phrases “attached to” or “attached directly to” refer to interaction between two or more entities which are in direct contact with each other and/or are separated from each other only by a fastener of any suitable variety (e.g., mounting hardware or an adhesive).

The terms “proximal” and “distal” are opposite directional terms. For example, the distal end of a device or component is the end of the component that is furthest from the practitioner during ordinary use. The proximal end refers to the opposite end, or the end nearest the practitioner during ordinary use.

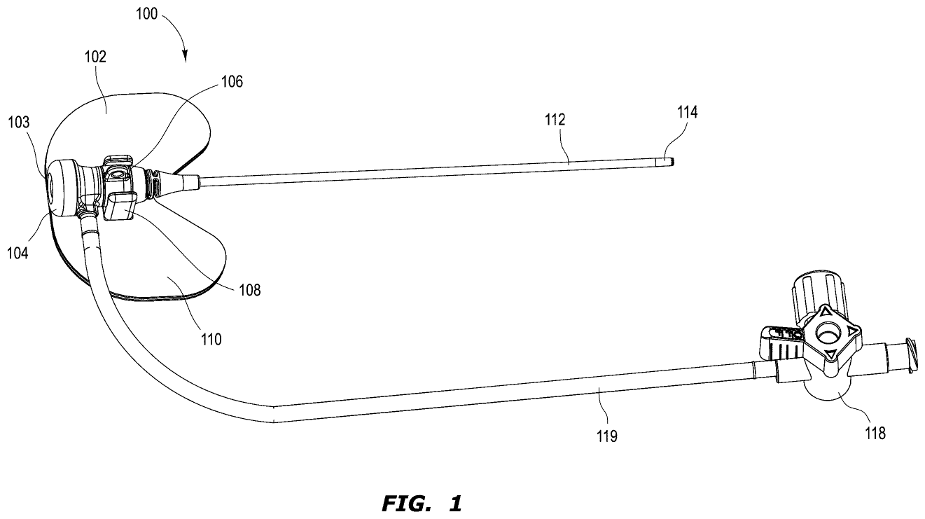

FIG. 1 illustrates a perspective view of an introducer system 100, according to some embodiments of the present disclosure. In the illustrated embodiment, introducer system 100 includes an introducer 103, a stopcock 118 fluidly connected to the introducer 103, and a sheath holder or an attachment assembly 102.

Introducer 103 can be used to introduce medical instruments into the vasculature of a patient. For instance, a distal end 114 of a sheath 112 of the introducer 103 can be inserted into a patient's vasculature through an incision or access site, while an introducer hub 104 of introducer 103 can remain exterior to the patient. Conventionally, introducer hub 104 can remain loose, or unattached, during medical procedures, which can lead to complications.

Accordingly, aspects and implementations of the present disclosure are directed to attachment assembly 102 for securing or fixing introducer hub 104 while providing additional stability and integrity to the introducer system 100. Stopcock 118 can be fluidly connected to introducer hub 104 via a fluid connecter 119.

In some embodiments, attachment assembly 102 can include a clip 108 and a base (e.g., adhesion component 110). A lower surface of the base is configured to secure clip 108 to an exterior surface of a patient's body. Clip 108 is configured to engage with an attachment portion 106 of introducer hub 104 to fix or anchor introducer hub 104 to the clip 108.

Clip 108 can be attached to adhesion component 110 which can in turn be adhesively attached to an anatomy, or an exterior portion of the patient's skin close to the access site. Fixation by adhesion component 110 can stabilize attachment assembly 102 and introducer system 100 at large.

In certain embodiments, the angle of insertion of sheath 112 into the patient's vasculature can vary. Thus, in some embodiments, clip 108 can be configured to fix, or hold introducer hub 104 at different heights, or offsets with respect to attachment assembly 102. For example, in some embodiments, clip 108 can hold introducer hub 104 (or sheath 112 or introducer 103) at a height, or offset, such that the angle made by sheath 112 at the point of insertion can be adjusted. For example, in some instances, the angle made by sheath 112 at the point of insertion can be anywhere between 20 and 65 degrees.

In some embodiments, clip 108 can include one or more securing points, for fixing, or securing a hub (e.g., through or at attachment portion 106 of the introducer hub 104). For instance, clip 108 can include multiple securing positions, each placed at different heights. Clip 108 may thus be configured with multiple securing positions for coupling with the attachment portion 106 of the introducer hub 104, such that disposition at different securing portions creates a different angle between the introducer and the patient's skin. Stated another way, different securing portions may be configured to accommodate a variety of angles at which sheath 112 enters or exits the body of a patient.

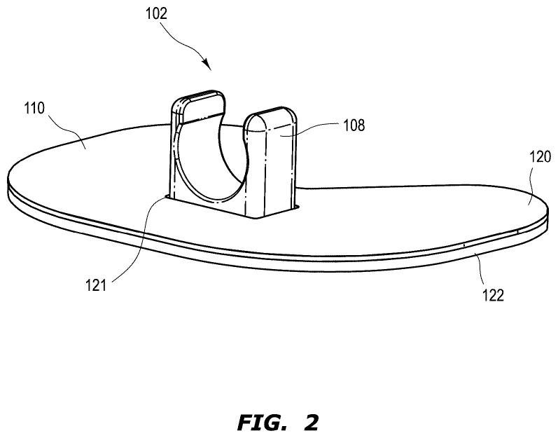

FIG. 2 illustrates a perspective view of the attachment assembly 102 of FIG. 1, according to embodiments described herein. In the illustrated embodiment, attachment assembly 102 includes clip 108 and adhesion component 110.

Adhesion component 110 includes top layer 120 and bottom layer 122. In some embodiments, top layer 120 and bottom layer 122 can together be configured to fix, or hold, clip 108 in place. For instance, in some embodiments, a portion of clip 108 can be placed between top layer 120 and bottom layer 122 of adhesion component 110. In the illustrated embodiment, top layer 120 and bottom layer 122 have a similar shape (e.g., perimeter) except that top layer 120 may include an aperture 121. Clip 108 can extend through aperture 121 in top layer 120. In such a way, clip 108 can be fixed to adhesion component 110.

Adhesion component 110, in turn, can be configured to adhesively fix clip 108 to an anatomical portion of the patient, or an exterior portion of the patient, close to the access site. This fixation can stabilize the attachment assembly and the introducer system at large.

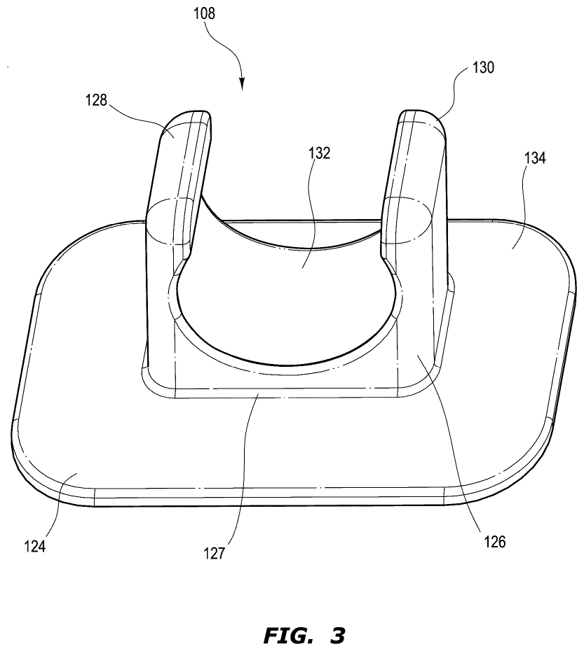

FIG. 3 illustrates a perspective view of the clip 108 of FIG. 1, according to embodiments described herein. In the illustrated embodiment, clip 108 includes a holding portion 126 and an anchor 124. As discussed above, adhesion component 110 can secure clip 108 to the skin of the patient. For example, bottom layer 122 can be fixedly attached to the exterior surface (e.g., skin) of the patient by an adhesive or the like. Anchor 124 of clip 108 may be placed on top of bottom layer 122. Holding portion 126 may be pass through aperture 121 of top layer 120 and top layer 120 can be secured to bottom layer 122 by an adhesive or the like. Due to the similar shapes of top layer 120 and bottom layer 122, top layer 120 can be aligned with bottom layer 122 to secure anchor 124 between the layers 120, 122.

Anchor 124 can be disposed between, or adhesively fixed between top layer 120 and bottom layer 122 of adhesion component 110 (as the components are seen in FIG. 2). In other words, anchor 124 is disposed internally within adhesion component 110. Holding portion 126 can extend or protrude from anchor 124. In some embodiments, holding portion 126 can include a pair of beams 128 and 130 which are configured to hold a sheath, hub, and/or attachment portion, of an introducer (as was seen with respect to FIG. 1). In some embodiments, holding portion 126 can include receptacle 132 which can be configured to receive and secure a sheath, hub, or attachment portion, of an introducer. Receptacle 132 can have a partially circular profile.

As discussed above, in certain embodiments, clip 108 includes multiple securing portions, or multiple points at which introducer hub 104 may be coupled to clip 108. In some such embodiments, beams 128, 130 may comprise multiple points, at various heights, configured to secure introducer hub 104. For example, inner walls of beams 128, 130 may include detents, undulations, recesses, and/or ridges configured to engage introducer hub 104 at various points along the height of beams 128, 130. In some cases, beams 128, 130 can be resilient.

In some embodiments, holding portion 126 and/or receptacle 132 can be configured to fix or secure a sheath or attachment portion of an introducer via a mechanism such as an interference, or a snap fit. In some embodiments, the sheath or attachment portion held within receptacle 132 can be configured to slide or translate longitudinally within receptacle 132, while still remaining fixed or secured with respect to other dimensions. Holding portion 126 and/or receptacle 132 can envelop a portion of an outer circumference of a sheath or hub of the introducer system.

In certain embodiments, beams 128, 130, holding portion 126, and/or receptacle 132 may be coupled to anchor 124 in a flexible or hinged arrangement. For example, holding portion 126 may be operably coupled to anchor 124 along a leading edge 127 in a such that the leading edge 127 comprises a hinge, a living hinge, a compliant interface, or other mechanism configured to flex and/or allow rotation of holding portion 126 with respect to anchor 124. In such embodiments, holding portion 126 may thus be able to be displaced with respect to anchor 124 via rotation about leading edge 127, allowing holding portion 126 to be disposed at an angle relative to anchor 124. This may allow for holding portion 126 to accommodate a wide range of angles at which a medical instrument (e.g., such as a sheath of an introducer system) enters or exits the body of the patient.

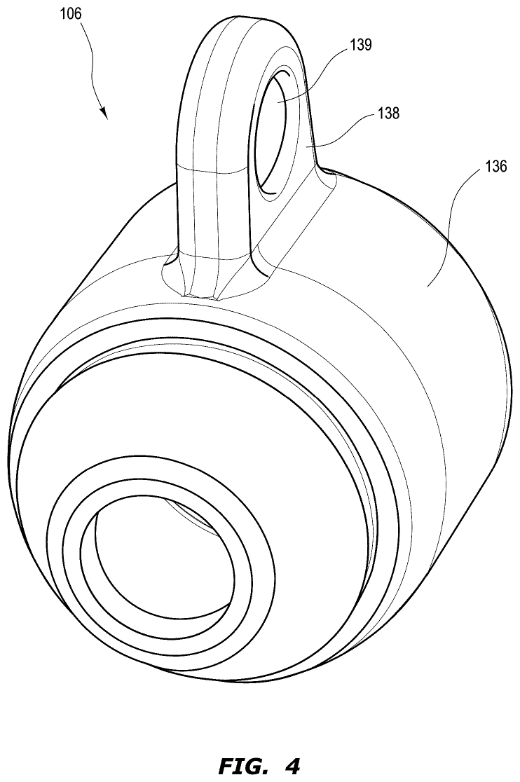

FIG. 4 illustrates attachment portion 106 of the introducer system of FIG. 1, according to embodiments described herein. In the illustrated embodiment, the attachment portion 106 includes a circular face 136 configured to engage with receptacle 132 or holding portion of clip 108 (as seen in FIG. 3). This may secure, or fix, the attachment portion 106 of introducer 103 to clip 108 of attachment assembly 102. Attachment portion 106 may further include a suture clip 138 that extends radially outward from circular face 136. The suture clip 138 may include an aperture 139. The attachment portion 106 may be further secured by the suture clip 138. For example, a first end of a suture may be secured to aperture 139 and a second end of the suture may be secured to the patient or another anchor.

In alternate embodiments, a shaft or sheath of the introducer may be configured to engage with the receptacle or holding portion of a clip. This may secure, or fix, the introducer and/or components thereof.

With reference to FIG. 1 and FIG. 4, in certain embodiments, introducer hub 104 may be configured such that introducer hub 104 can be rotated about its longitudinal axis when fixed or attached to clip 108. Otherwise stated, clip 108 can resist longitudinal displacement of the introducer hub 104, while allowing for rotation of the introducer hub 104 about its longitudinal axis. Suture clip 138 may limit rotation of introducer hub 104 because suture clip 138 engage with beams 128, 130.

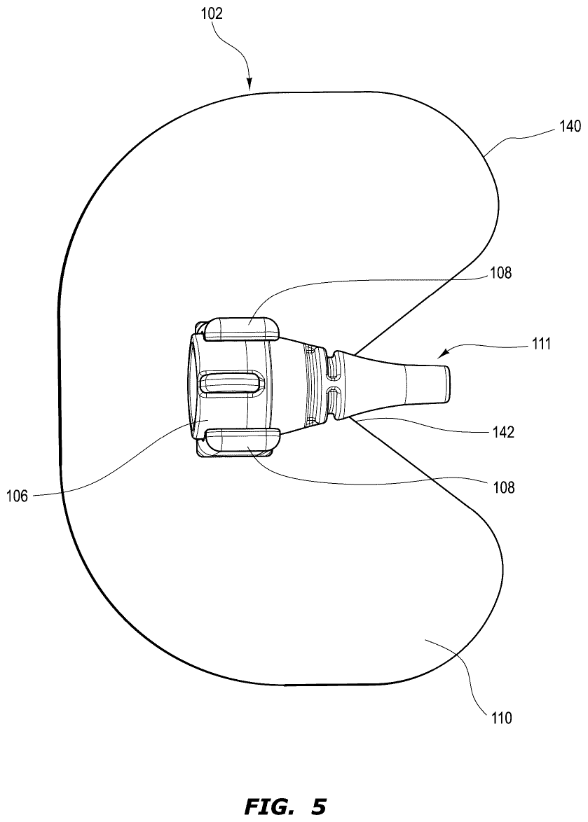

FIG. 5 illustrates a top-down view of attachment assembly 102 and attachment portion 106 of introducer system 100 of FIG. 1, according to embodiments disclosed herein. In the illustrated embodiment, attachment assembly 102 includes a recess 111 within distal edge 140 of adhesion component 110. As discuss earlier, top layer 120 and bottom layer 122 have a similar shape. Accordingly, top layer 120 and bottom layer 122 both comprise recess 111 that align with each other when top layer 120 is attached to bottom layer 122.

Recess 111 can be configured to accommodate an entry site or insertion site into the body. For instance, in some embodiments, adhesion component 110 and/or recess 111 can be shaped such that portion of adhesion component 110 extends along the sides or periphery of an access site (e.g., which would be to the right of adhesion component 110 as seen in FIG. 5). In some embodiments, were adhesion component 110 to be absent of recess 111, the nearest point of adhesion component 110 to an insertion point for the introducer would be on an exterior edge of adhesion component 110, along an axis formed by the introducer 103 or clip 108 projected onto the surface of adhesion component 110. Thus, including recess 111 can provide additional distance to the insertion site, while maintaining a large adhesion surface. Thus, recess 111 can facilitate securing of adhesion component 110 to the body at a location proximate to the entry site, without interfering with the entry site.

In some embodiments, recess 111 can be a space or recess that is spaced from any possible fluids that leak from the insertion site. Recess 111 can extend inwardly along an axis of a receptacle of the clip 108. Otherwise stated, recess 111 can facilitated and maintain the integrity of the adhesive bond between the adhesion component 110 and the exterior surface and/or skin of a patient.

As seen in FIG. 5, recess 111 can limit the distance that adhesion component 110 extends in the distal direction. In some embodiments, recess 111 can be disposed or correspond to a central axis of clip 108, or the receptacle(s) 132 within clip 108. For instance, in some embodiments, recess 111 can extend inwardly along a projection of a central axis of receptacle 132 of the clip 108. In some cases, recess 111 can conform to a projection of a central axis of the receptacle onto a plane defined by adhesion component 110 (e.g., onto the exterior surface and/or skin of a patient). For instance, in some embodiments, recess 111 can be configured to maintain a minimum predefined distance between an exterior edge (e.g., distal edge 140) of adhesion component 110 and an insertion site associated with an introducer system secured by receptacle 132.

In some embodiments, recess 111 can encircle, or extend around, a perimeter of an insertion site or point. Accordingly, recess 111 can extend from distal edge 140 to a proximal-most portion 142.

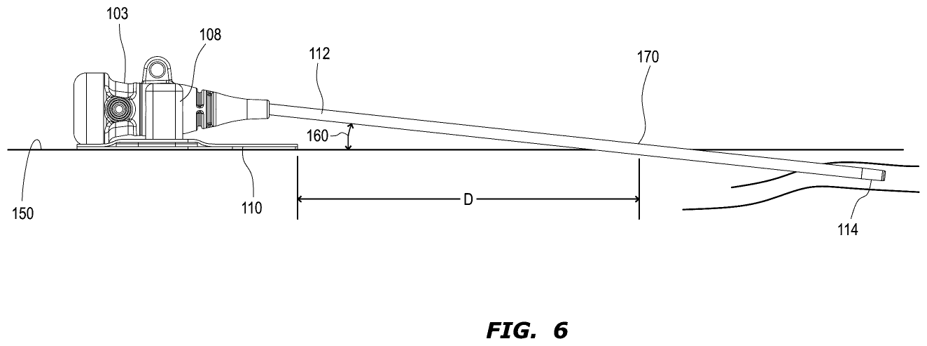

FIG. 6 illustrates a lateral view of an insertion point 170 for the introducer system 100 of FIG. 1, according to embodiments disclosed herein. In the illustrated embodiment, sheath 112 is seen extending from clip 108 to insertion point 170. As seen, distal end 114 of sheath 112 and/or introducer 103 passes subdermally into the vasculature of the patient (e.g., through the skin 150 or into the body of a patient). Insertion point 170 can be spaced a distance D from the distal-most edge, (e.g., distal edge 140 of FIG. 5).

The distance D, together with a height of clip 108 with respect to the skin 150 of a patient, can define or influence an angle of insertion 160. Otherwise stated, an angle of insertion 160 can be the angle formed by the offset of clip 108 (and/or the receptacle) relative to an exterior surface (e.g., the patient's skin) at insertion point 170.

As seen in FIG. 6, a higher distance, or offset, of clip 108 with respect to adhesion component 110 can increase the angle of insertion 160. For instance, in some embodiments, clip 108 (and/or a corresponding receptacle) can be offset from the adhesion component 110 at a distance that forms an angle between about 30 degrees to about 50 degrees relative to the exterior surface of the patient's skin at an insertion site. For instance, in some embodiments, the angle of insertion 160 (“insertion angle”) formed by introducer system 100 at an insertion site (e.g., insertion point 170) is anywhere between about 30 to about 50 degrees.

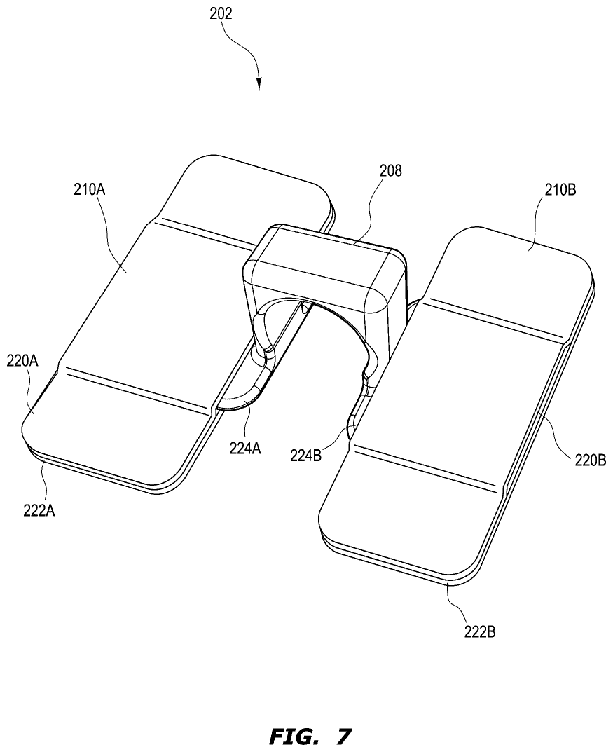

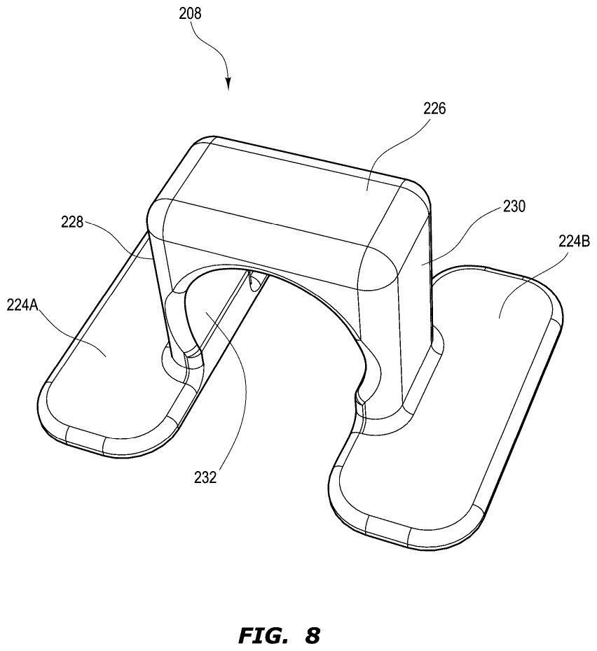

FIGS. 7 and 8 illustrate an embodiment of an attachment assembly 202 and clip 208 that resembles the attachment assembly 102 and clip 108 described above in certain respects. Accordingly, like features are designated with like reference numerals, with the leading digits incremented to “2”. For example, the embodiment depicted in FIG. 8 includes clip 208 that may, in some respects, resemble clip 108 of FIGS. 1-3. Relevant disclosure set forth above regarding similarly identified features thus may not be repeated hereafter. Moreover, specific features of attachment assembly 102 and clip 108 and related components shown in FIGS. 1-3 may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of attachment assembly 202 and clip 208 and related components depicted in FIGS. 7 and 8.

Any suitable combination of the features, and variations of the same, described with respect to attachment assembly 102, clip 108, and related components illustrated in FIGS. 1-3 can be employed with attachment assembly 202, clip 208 and related components of FIGS. 7 and 8, and vice versa. This pattern of disclosure applies equally to further embodiments depicted in subsequent figures and described hereafter, wherein the leading digits may be further incremented.

FIG. 7 illustrates attachment assembly 202 for use with the introducer system 100 of FIG. 1, according to embodiments described herein. In the illustrated embodiment, attachment assembly 202 can be configured to secure an introducer component (as seen in FIG. 1). As seen, attachment assembly 202 includes clip 208 and a base. The base includes a first adhesion component 210A, and a second adhesion component 210B. A lower surface of the base is configured to secure clip 208 to an exterior surface of a patient's body.

First adhesion component 210A can include a top layer 220A and a bottom layer 222A. The top layer 220A and the bottom layer 222A can have a similar shape so that top layer 220A can substantially align with bottom layer 222A when top layer 220A adheres to bottom layer 222A. Second adhesion component 210B can include a top layer 220B and a bottom layer 222B. The top layer 220B and the bottom layer 222B can have a similar shape so that top layer 220B can substantially align with bottom layer 222B when top layer 220B adheres to bottom layer 222B.

In some embodiments, top layers 220A, 220B and bottom layers 222A, 222B can together be configured to fix, or hold, clip 208 in place. In some embodiments, adhesion component 210 can be configured to adhesively fix the clip 208 to an anatomical portion of the patient, or an exterior portion of the patient, close to the access site. This fixation can serve to stabilize attachment assembly 202 and the introducer system at large.

As previously discussed, in some embodiments, the angle of insertion of a sheath into the patient's vasculature can vary, in some embodiments, clip 208 can be configured to fix, or hold an introducer hub at different heights, or offsets with respect to adhesion components 210A, 210B. For example, in some embodiments, clip 208 can hold a hub at a height, or offset, such that the angle made by the sheath at the point of insertion is anywhere between 20 through 65 degrees. Accordingly, in some embodiments, clip 208 can include one or more securing points, for fixing, or securing a hub, each placed at different heights. Additionally, clip 208 may be configured with hinges, compliant members, or other features that allow clip 208 to rotate to different angles with respect to the body and adhesion components 210A, 210B.

The adhesion components 210A, 210B may be shaped such that they may be disposed about a periphery of an access site, without interfering with the access site.

The embodiment shown in FIG. 7 to FIG. 8 may be configured such that clip 208 may be pushed down onto an access sheath or catheter without having to dislodge the sheath and/or navigate any portion of attachment assembly 202 under the sheath or catheter.

FIG. 8 illustrates a perspective view of clip 208 of attachment assembly 202 of FIG. 7, according to embodiments described herein. In the illustrated embodiment, clip 208 includes a holding portion 226 and anchors 224A, 224B.

Anchors 224A, 224B can be sandwiched, disposed, or adhesively fixed, between top layers 220A, 220B and bottom layers 222A, 222B of adhesion components 210A, 210B (as the components are seen in FIG. 7). For instance, in some embodiments, anchor 224A can be sandwiched, or adhesively fixed between top layers 220A and bottom layers 222A (of FIG. 7). Anchor 224B can be sandwiched, or adhesively fixed between top layers 220B and bottom layers 222B (of FIG. 7).

As discussed above, adhesion component 210 can secure clip 208 to the skin of the patient. For example, bottom layers 222A, 222B can be fixedly attached to the exterior surface (e.g., skin) of the patient by an adhesive or the like. Bottom layers 222A, 222B are laterally spaced apart. Anchors 224A, 22B of clip 108 may be placed on top of bottom layers 222A, 222B. Top layers 220A, 220B can be secured to bottom layers 222A, 222B by an adhesive or the like with anchors 224A, 224B partially disposed between top layers 220A, 220B and bottom layers 222A, 222B. Due to the similar shapes of top layers 220A, 220B and bottom layers 222A, 222B, top layers 220A, 220B can be aligned with bottom layers 222A, 222B to secure anchors 224A, 224B between the layers 220A, 220B, 222A, 222B.

Holding portion 226 can extend or protrude from anchors 224A, 224B. In some embodiments, holding portion 226 can include a pair of beams 228 and 230 which are configured to hold a sheath or attachment portion, of an introducer component (as was seen with respect to FIG. 1). In some embodiments, holding portion 226 can include a receptacle 232 which can be configured to receive and secure a sheath or attachment portion, of an introducer component.

In some embodiments, holding portion 226 and/or receptacle 232 can be configured to fix or secure a sheath or attachment portion of an introducer via a mechanism such as an interference, or a snap fit. In some embodiments, the sheath or attachment portion held within receptacle 232 can be configured to slide or translate longitudinally within the receptacle 232, while remaining fixed or secured with respect to other dimensions.

Additionally, in some embodiments, holding portion 226 of clip 208 can be configured to open, or disengage from one or both anchors (e.g., 224A and/or 224B), to facilitate removal and/or insertion of a hub or shaft of an introducer component into/from receptacle 232. For example, either beam 228 and/or 230 can be operatively connected to anchors 224A and/or 224B (respectively) such that the clip 208 can be transitioned into an open configuration, or into a closed configuration. (The closed configuration is seen in FIG. 8).

In some embodiments, this operative connection can include a living hinge, a snap-fit, a fastener, or any other kind or type of operative coupling. In some embodiments, a living hinge may be disposed on one beam (e.g., beams 228) and a detent or catch on the opposing beam (e.g., beam 230).

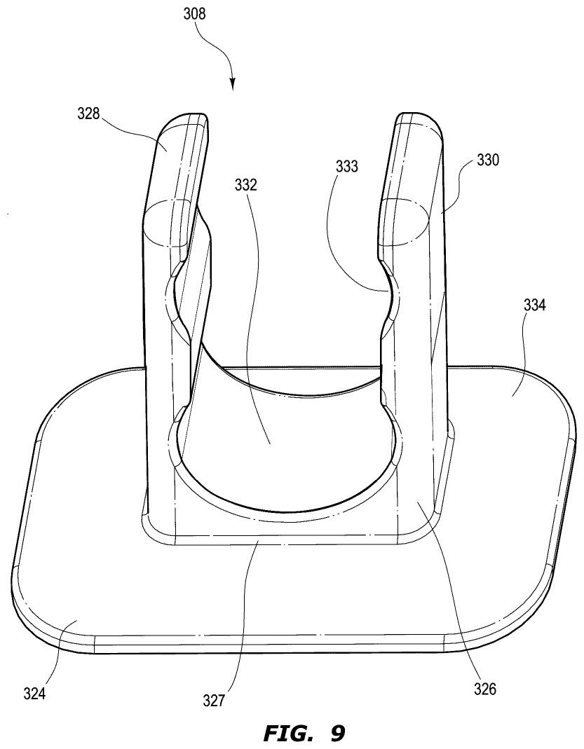

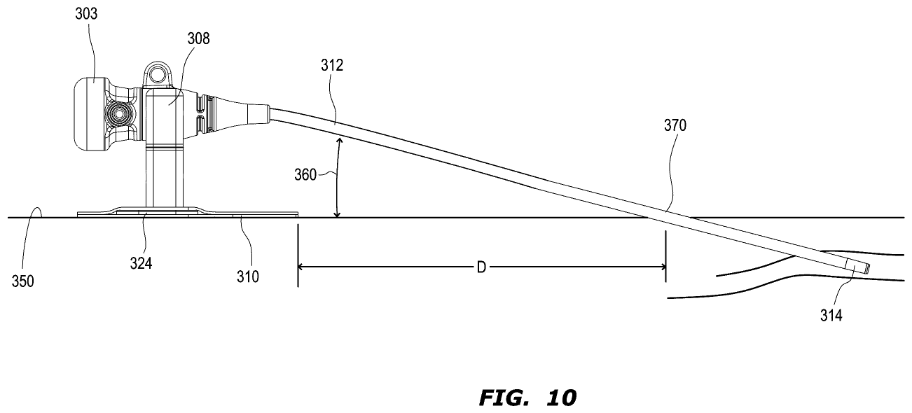

FIGS. 9 and 10 illustrates an embodiment of an attachment assembly 302. FIG. 9 illustrates a perspective view of a clip 308 for use with the introducer system of FIG. 1, according to embodiments described herein. In the illustrated embodiment, clip 308 includes a holding portion 326 including multiple receptacles 332, 333 for holding an introducer, and an anchor 324.

As shown, holding portion 326 can extend or protrude from anchor 324. In some embodiments, holding portion 326 can include beams 328, 330 configured to hold a sheath, hub, and/or attachment portion, of an introducer (as was seen with respect to FIG. 1 and/or FIG. 3). In some embodiments, holding portion 326 can include receptacles 332, 333 which can be configured to receive and secure a sheath, hub, or attachment portion, of an introducer at different heights relative to the anchor 324.

Similar to the discussion provided with respect to FIG. 1 and/or FIG. 3, in certain embodiments, clip 308 includes multiple securing portions, or multiple points at which an introducer hub can be coupled to the clip 308. For instance, in some such embodiments, beams 328, 330 can include multiple points, at various heights (e.g., at receptacles 332, 333) configured to secure an introducer hub. For example, the inner walls of beams 328, 330 can include receptacle 333 which can be disposed vertically above, or at a height higher than receptacle 332. Accordingly, clip 308 can hold or fix an introducer or introducer hub at various points along the height of beams 328, 330. In some embodiments, clip 308 can include more than two receptacles (e.g., beams 328, 330 can extend and/or include additional mechanisms and/or points to hold an introducer.

As previously discussed, holding portion 326 and/or receptacles 332, 333 can be configured to fix or secure a sheath or attachment portion of an introducer via a mechanism such as an interference, or a snap fit. This may allow for holding portion 326 to accommodate a medical instrument (e.g., such as a sheath of an introducer system) at one or more vertical heights or offsets from anchor 324. This can allow for holding portion 326 to accommodate a medical instrument (e.g., such as a sheath of an introducer system) with a wide range of angles at which a medical instrument (e.g., such as a sheath of an introducer system) enters or exits the body of the patient.

FIG. 10 illustrates a lateral view of an insertion point 370 and an introducer system 300 including clip 308 of FIG. 9, according to embodiments disclosed herein. In the illustrated embodiment, a sheath 312 is seen extending from clip 308 to insertion point 370. As seen, an introducer 303 is held at a higher offset or vertical distance with respect to adhesion component 310. A distal end 314 of sheath 312 and/or introducer 303 passes subdermally into the vasculature of the patient (e.g., through the skin 350 or into the body of a patient). Insertion point 370 can be spaced a distance D from the distal-most edge, (e.g., distal edge 340 of FIG. 9).

The distance D, together with a height that introducer 303 is being held within clip 308 with respect to the skin 350 of a patient, can define or influence an angle of insertion 360. Otherwise stated, an angle of insertion 360 can be the angle formed by the offset of clip 308 (and/or the receptacle) relative to an exterior surface (e.g., the patient's skin) at insertion point 370.

As seen in FIG. 10, a higher distance, or offset, of clip 308 with respect to adhesion component 310 can increase the angle of insertion 360. For instance, in some embodiments, clip 308 (and/or a corresponding receptacle) can be offset from adhesion component 310 at a distance that forms an angle between about 30 degrees to about 50 degrees relative to an exterior surface of the patient's skin at an insertion site. For instance, in some embodiments, the angle of insertion 360 (“insertion angle”) formed by the introducer system at an insertion site (e.g., insertion point 370) is anywhere between about 30 to about 50 degrees.

Any methods disclosed herein include one or more steps or actions for performing the described method. The method steps and/or actions may be interchanged with one another. In other words, unless a specific order of steps or actions is required for proper operation of the embodiment, the order and/or use of specific steps and/or actions may be modified. Moreover, sub-routines or only a portion of a method described herein may be a separate method within the scope of this disclosure. Otherwise stated, some methods may include only a portion of the steps described in a more detailed method.

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

Similarly, it should be appreciated by one of skill in the art with the benefit of this disclosure that in the above description of embodiments, various features are sometimes grouped together in a single embodiment, figure, or description thereof for the purpose of streamlining the disclosure. This method of disclosure, however, is not to be interpreted as reflecting an intention that any claim requires more features than those expressly recited in that claim. Rather, as the following claims reflect, inventive aspects lie in a combination of fewer than all features of any single foregoing disclosed embodiment. Thus, the claims following this Detailed Description are hereby expressly incorporated into this Detailed Description, with each claim standing on its own as a separate embodiment. This disclosure includes all permutations of the independent claims with their dependent claims.

Recitation in the claims of the term “first” with respect to a feature or element does not necessarily imply the existence of a second or additional such feature or element. It will be apparent to those having skill in the art that changes may be made to the details of the above-described embodiments without departing from the underlying principles of the present disclosure.

We claim:

1. A sheath holder, comprising:
a clip comprising:
a first receptacle configured to secure a sheath or hub of an introducer system to the clip; and
a first anchoring portion; and
a base configured to secure the first anchoring portion of the clip to an exterior surface of a body of a patient.

2. The sheath holder of claim 1, wherein the first receptacle comprises a structure including a partially circular profile; and
wherein the structure is configured to envelop a portion of an outer circumference of a sheath or hub of the introducer system.

3. The sheath holder of claim 1, wherein the clip comprises a pair of resilient beams configured to snap fit around a portion of an outer circumference of the sheath or the hub of the introducer system.

4. The sheath holder of claim 1, wherein the base comprises an aperture configured to receive the clip.

5. The sheath holder of claim 1, wherein the base comprises a recess extending inwardly along an axis of the first receptacle.

6. The sheath holder of claim 1, wherein the clip further comprises a second anchoring portion; and
wherein the base is configured to secure the clip via the first anchoring portion and the second anchoring portion to the exterior surface of the body of the patient.

7. The sheath holder of claim 1, wherein the first receptacle is offset from the base at a distance that forms an angle between about 30 degrees to about 50 degrees relative to the exterior surface of the body of the patient at an insertion site.

8. The sheath holder of claim 1, wherein the sheath holder is configured to support the introducer system such that an insertion angle formed by the introducer system at an insertion site is anywhere between about 30 to about 50 degrees.

9. The sheath holder of claim 1, wherein the clip comprises a hinge mechanism configured to operatively close and open to secure the sheath or the hub of the introducer system.

10. The sheath holder of claim 1, wherein the clip comprises a second receptacle that is vertically offset from the first receptacle.

11. The sheath holder of claim 1, wherein the base comprises at least one of:
a recess conforming to a projection of a central axis of the first receptacle onto a plane defined by the base; or
a recess configured to maintain a minimum predefined distance between an exterior edge of the base and an insertion site associated with an introducer system secured by the receptacle.

12. A sheath holder, comprising:
a clip comprising:
a receptacle configured to secure a sheath or hub of an introducer system; and
an anchoring portion; and
a base comprising:
a lower surface configured to secure the anchoring portion of the clip to an exterior surface of a patient's body; and
a recess conforming to a projection of a central axis of the receptacle onto a plane defined by the base.

13. The sheath holder of claim 12, wherein the receptacle comprises a structure including a partially circular profile; and
wherein the structure is configured to envelop a portion of an outer circumference of the sheath or the hub of the introducer system.

14. The sheath holder of claim 12, wherein the anchoring portion is disposed internally within the base.

15. The sheath holder of claim 12, wherein the receptacle is vertically offset from the base at a height that forms an angle between about 30 degrees to about 50 degrees relative to the exterior surface of the body of the patient at an insertion site.

16. A sheath holder, comprising:
a clip comprising:
a receptacle configured to secure a sheath or hub of an introducer system; and
a first anchoring portion; and
a base comprising:
a lower surface configured to secure the clip via the first anchoring portion to an exterior surface of a body of a patient; and
a recess configured to maintain a minimum predefined distance between an exterior edge of the base and an insertion site associated with the introducer system secured by the receptacle.

17. The sheath holder of claim 16, wherein the receptacle comprises a structure including a partially circular profile; and
wherein the structure is configured to envelop a portion of an outer circumference of a sheath or hub of the introducer system.

18. The sheath holder of claim 16, wherein the clip comprises a pair of resilient beams configured to snap fit around a portion of an outer circumference of the sheath or hub of the introducer system.

19. The sheath holder of claim 16, wherein the receptacle is vertically offset from the base at a height that forms an angle between about 30 degrees to about 50 degrees relative to an exterior surface of the body of the patient at the insertion site.

20. The sheath holder of claim 16, wherein the sheath holder is configured to support the introducer system such that an insertion angle formed by the introducer system at the insertion site is anywhere between about 30 to about 50 degrees.
